(19) United States
(12) Patent Application Publication
(10) Pub. No.: US 2020/0374641 A1
(43) Pub. Date: Nov. 26, 2020

(54) HEARING SYSTEM WITH A HEARING INSTRUMENT

(71) Applicant: SIVANTOS PTE. LTD., SINGAPORE (SG)

(72) Inventor: KUNIBERT HUSUNG, ERLANGEN (DE)

(21) Appl. No.: 16/878,776

(22) Filed: May 20, 2020

(30) Foreign Application Priority Data
May 20, 2019 (DE) 102019207373

(51) Int. Cl. H04R 25/00; A61B 5/0402

(52) U.S. Cl. CPC H04R 25/609; A61B 5/0402; H04R 25/70; H04R 25/505

(57) ABSTRACT

A hearing system contains a hearing instrument with a first sensor electrode, a second sensor electrode and an evaluation unit. The evaluation unit is set up to establish an electrocardiogram of a wearer of the hearing instrument using a first sensor signal, which is output by the first sensor electrode, and a second sensor signal, which is output by the second sensor electrode, if the hearing instrument is worn as intended by the wearer and the first sensor electrode and the second sensor electrode are each in contact with a body part of the wearer.

### CROSS-REFERENCE TO RELATED APPLICATION

This application claims the priority, under 35 U.S.C. § 119, of German application DE 10 2019 207 373, filed May 20, 2019; the prior application is herewith incorporated by reference in its entirety.

### BACKGROUND OF THE INVENTION

### Field of the Invention

The invention relates to a hearing system with a hearing instrument and an evaluation unit.

In a hearing aid, an electro-acoustic input transducer, which is usually, represented by a microphone, coverts an ambient sound into a corresponding electrical signal, which is then processed depending on a hearing impairment of the wearer of the hearing aid which is to be corrected and is amplified in particular depending on the frequency band. The output signal which results from signal processing is delivered to the ear of the wearer by a sound generator of the hearing aid, which can be present in the form of a speaker or also a bone conduction earphone. During signal processing, it is also possible to carry out noise suppression, acoustic feedback suppression, to highlight certain sound sources such as an interlocutor, for example, by directional microphone technology or other processes for improving the quality of the output signal, in particular with respect to its useful signal component.

Hearing impairments which are intended to be corrected for a wearer by a hearing aid occur in particular in older people who are often showing signs of age-related hearing loss in higher frequency bands, whereby in the case of the affected individuals, acoustic speech comprehension, i.e. the capacity for acoustic resolution of speech, is compromised as a result of the natural frequency spectrum of speech. However, older people often also have further medical problems which may necessitate regular monitoring.

On the other hand, the increasing miniaturization of electronic components such as sensors, but also components for data transmission, is now already resulting in an extension of the range of functions of hearing instruments such as hearing aids, but also earphones which are used by individuals with normal hearing, for example to listen to music or in connection with mobile phones. In particular, hearing aids are also being wirelessly coupled to external devices such as mobile phones in order to select a particular hearing program for a specific hearing situation, or to be able to vary sound settings directly.

### BRIEF SUMMARY OF THE INVENTION

The underlying object of the invention is to specify a hearing system with a hearing instrument which has an extended range of functions and in this case in particular is set up to generate medically relevant information.

The stated object is achieved according to the invention by a hearing system, containing a hearing instrument with a first sensor electrode and a second sensor electrode and an evaluation unit. The evaluation unit is set up to establish an electrocardiogram (ECG) of a wearer of the hearing instrument using a first sensor signal, which is output by the first sensor electrode, and a second sensor signal, which is output by the second sensor electrode, if the hearing instrument is worn as intended by the wearer and the first sensor electrode and the second sensor electrode are each in contact with a body part of the wearer. Configurations which are advantageous and are themselves partly inventive are the subject matter of the subclaims and the following description.

In this case, a hearing instrument contains in particular a hearing aid for correcting a hearing impairment of a wearer as well as an earphone for reproducing an audio signal, wherein, in this case, the earphone does not need to have any independent functions for correcting hearing impairments.

In this case, the evaluation unit, on the one hand, can be incorporated into the hearing instrument as part of the hearing instrument, i.e. in particular it can be arranged inside a housing of the hearing instrument which is worn on or at least partially in one ear by the wearer. In the case of a hearing aid, the essential elements for generating and processing the signal which are specific to the hearing aid are also incorporated into the housing, i.e. in particular at least one electro-acoustic input transducer and one signal processing unit which is connected to the input transducer. In the case of an evaluation unit which is incorporated into the hearing instrument, the evaluation unit is preferably connected to the first sensor electrode and the second sensor electrode respectively.

Alternatively, the evaluation unit can be part of an external housing, with respect to the hearing instrument, i.e. part of a remote control or a mobile phone with a corresponding mobile application (“app”), for example. In this case, the hearing instrument is preferably set up to transmit the first sensor signal and the second sensor signal to the external device which contains the evaluation unit, for example by means of radio frequency transmission or Bluetooth.

In this case, the first sensor electrode and the second sensor electrode are configured according to their structure to generate, when in corresponding contact with a body part of the wearer of the hearing instrument, a first or second sensor signal in such a way that the first sensor signal and the second sensor signal are in principle suitable for establishing an ECG from the information content. In particular, the first sensor electrode and the second sensor electrode are set up to measure an electrical voltage or potential difference and/or the change in such a voltage or potential difference. In this case, the first and the second sensor electrode are preferably applied to the surface of the housing of the hearing instrument or are incorporated therein in such a way that the described measurement of a voltage on a body part which touches the housing at the corresponding point of the relevant sensor electrode is technically possible, i.e. the sensor signals have a signal strength which is sufficient for establishing an ECG.

In operation, the hearing instrument is worn as intended by the wearer on the ear or is at least partially inserted into an ear canal, so that an output sound generated by the hearing instrument can be delivered to the ear of the wearer by way of a sound generator of the hearing instrument. In this case, intended wearing is defined in particular by the form of the hearing instrument as well as, where applicable, by the position of its input transducer or its input transducer and its sound generator. In this case, in particular, contact with a body part of the wearer is established already at least for one sensor electrode by the hearing instrument being worn as intended, in other words by its positioning, for example with the skin at the entrance of the ear canal, at the concha or in the region of the auricle.

In this case, the ECG can be initiated by the wearer actively establishing contact of a body part, for example a finger, with the second sensor electrode. However, commencing an ECG can also take place by selecting a corresponding function on the hearing instrument itself, or in a corresponding app or the like (in the case of a mobile phone as an external device).

The arrangement of the two sensor electrodes in the hearing instrument is preferably such that, by way of corresponding contact with body parts and points of the body of the wearer via the sensor electrodes, an electrical circuit can be closed which leads through the heart of the wearer, so that the ECG signal is particularly strong since it does not need to be derived from secondary voltages in the body but rather directly records the electrical activity of the heart.

In an electrocardiogram, electrical voltage changes on the surface of the body are measured which are caused by electrical excitation of the heart. These electrical excitations are spread over the heart muscle in the so-called center for producing excitation via the so-called excitation control system of the heart, in order to stimulate the contractions thereof and thus the pumping activity for the blood circulation. In repose, the heart muscle cells thus generally have a negative membrane potential, i.e. the extracellular space is positively charged, while this relation is reversed in the case of electrically excited cells. The electrical voltage, which is caused by an arising charge distribution in the extracellular space of the heart, can be measured on the skin. As a result, the periodic contractions of the heart can be verified and checked via the electrical activity on the heart which in each case precedes them and causes them. In particular, deviations from the contraction patterns which, medically speaking, are normal, can here be recognized.

The present invention permits an ECG to be incorporated into a hearing instrument, such that monitoring individual heart activity is made possible in a particularly simple manner and without the need for additional devices, especially for hearing-impaired individuals who moreover are also suffering from occasional or chronic heart problems, as is more often the case for older individuals in particular.

The first sensor electrode is preferably arranged in the hearing instrument in such a way that it is readily accessible when worn as intended. In the event that the first sensor electrode is not located on the surface of the housing, but instead is incorporated into it, this means that the corresponding point on the housing is readily accessible. In particular, this has the result that, when worn as intended, the wearer is able to touch with a finger the point in the housing of the hearing instrument at which the first sensor electrode is arranged on the surface or is incorporated into the housing, and apart from that, this point is not in direct contact with the body of the wearer without the wearer actively intervening in the form of a finger and/or hand movement. This makes it possible to carry out an ECG measurement on a bioelectrical circuit which leads to the head via the arm of the wearer and thus past the heart, whereby the potential differences which arise at the heart can be measured particularly easily.

In this case, the evaluation unit is conveniently set up to establish the ECG of the wearer if the wearer touches with a part of the hand a point on the hearing instrument at which the first sensor electrode is arranged. In this case, the wearer preferably touches the hearing instrument with one finger or touches the hearing instrument with another part of the same hand on the side on which the hearing instrument is also worn. In particular, the hearing instrument is worn on or at least partially in the left ear, wherein the wearer touches the relevant point on the hearing instrument with one finger or another part of the left hand for an ECG measurement. This is a movement which can be easily performed by the wearer and which achieves an advantageous bioelectrical circuit for the ECG.

Advantageously, the hearing system further contains a visualization means, wherein the evaluation unit is set up to transmit an established ECG to the visualization means, and wherein the visualization means is set up to graphically display a received ECG. In particular, the visualization means can also be implemented by a mobile phone with a corresponding app for communicating with the hearing instrument for the purpose of transmitting the ECG and graphically displaying it, or an independent remote control specifically for the hearing instrument. The wearer preferably carries the entire hearing system when it is used as intended, which is specified by the intended use of the hearing instrument and the corresponding application of the visualization means. The visualization of an ECG by way of the visualization means permits the wearer themselves to initially check the ECG for possible abnormalities, if necessary also individually and specifically for their specific medical status.

The evaluation unit is here advantageously incorporated into the visualization means. In this case, in particular, the first sensor signal and the second sensor signal are transmitted from the hearing instrument, preferably wirelessly, to the visualization means. The evaluation unit can here in particular also be implemented by a processing unit and a storage unit of a mobile phone as well as a corresponding app. As a result, the computational resources of the visualization means may be used without straining the battery of the hearing instrument beyond establishing the sensor signals for an ECG. The ECG is transmitted from the evaluation means to the visualization means if the evaluation means is incorporated into the visualization means, in particular via corresponding interfaces on hardware and/or software levels, so that the region of the visualization means which is used for evaluating the sensor signals outputs the established ECG to a region which is responsible for the visualization.

In one further advantageous, alternative configuration, the evaluation unit is incorporated into the hearing instrument. This permits only a complete ECG waveform to be transmitted to the visualization means for displaying, or it also permits a visualization to be dispensed with and the ECG to be checked in the hearing instrument for abnormalities, wherein a warning measure or the like is only taken if an abnormality is ascertained.

It proves to be advantageous if the hearing instrument is configured as an earphone. The wearer uses an earphone to reproduce an audio signal, wherein an output sound which is generated by a sound generator of the earphone is directly introduced into the ear canal. In this case, the earphone has, where applicable, a signal processing unit which can generally be set up to amplify an input signal, which is to be reproduced, depending on the frequency band, wherein this amplification can usually be selected by the wearer based on their hearing preferences and is not typically used for correcting a hearing impairment of the wearer. In this case, earphones are used in the context of communication devices such as mobile phones, tablet PCs and the like, or with entertainment electronics such as mp3 players, for example. Since earphones are often also worn during sport or similar activities, a wearer is able to establish an ECG during a training session or break in a particularly simple manner.

It further proves to be advantageous if the hearing instrument of the hearing system is configured as a hearing aid which is provided and set up to correct a hearing impairment of a wearer. Consequently, when using the hearing aid, a further function is provided which is important for the wearer's health, is potentially lifesaving, and is particularly significant given the fact that hearing aids are often worn by older people.

In one advantageous configuration of the hearing system, the hearing instrument further comprises an earpiece, wherein the second sensor electrode is arranged in the earpiece, preferably on an outer surface. In this case, the concept of an earpiece contains in particular any component of a hearing instrument which is to be at least partially inserted into the ear canal or into the concha for the intended use, i.e. for a hearing instrument which is configured as a hearing aid, a dome which is arranged on a sound tube or a similar earmold in the case of a behind-the-ear aid (BTE), a hearing piece in the case of a receiver-in-the-canal aid (RIC), or a corresponding housing part of an in-the-ear aid (ITE), for example. If an earphone is a hearing instrument, the earpiece is formed through a part of the housing or the entire housing of the earphone. In the case of a sound tube of an RIC aid, the second sensor electrode is preferably arranged on the dome or on the earmold, wherein a first sensor cable leads along the sound tube for transmitting the second sensor signal to an evaluation unit or a communications unit in the housing. Arranging the second sensor electrode in an earpiece results in high stability against displacements, since the displacements are effectively prevented by the fixing effect of the ear canal or the concha.

It also proves to be advantageous if the hearing instrument which is configured as a hearing aid further contains a housing which is set up to be worn behind an auricle of the wearer, wherein the second sensor electrode is arranged in the housing, preferably facing the skin when worn. In particular, the hearing aid can be configured as a BTE aid or as an RIC aid. For hearing aids of this type, depending on the detailed configuration of a relevant earpiece, it can be more cost effective to dispense with a first sensor electrode in the earpiece and a corresponding first sensor cable along the earpiece, which is often reversibly connected to the housing, and instead to arrange the second sensor electrode on the part of the housing which is to be worn behind the auricle during operation and is thus also stabilized by this against displacements which could result in measurement errors in the ECG measurement. In this case, wearing it behind the auricle is intended to be understood to mean any arrangement in which at least one part of the housing is arranged behind the auricle from a lateral view of the ear.

Advantageously, the hearing instrument is further set up to measure a pulse of the wearer, wherein the evaluation unit is set up to determine a blood pressure using the measured pulse and the ECG. In particular, the measured pulse of the wearer is therefore transmitted from a measuring apparatus, provided for measuring the pulse, to the evaluation unit, or measurement data of a measuring apparatus of this type are transmitted to the evaluation unit, so that the pulse measurement is evaluated in the evaluation unit, in particular in an independent module of the evaluation unit which is specifically for pulse measurement and which is in communication with a module of the evaluation unit which is specifically for ECG measurement. The ECG measures heart activity substantially without significant delay, while a pulse measurement provides delayed information on the heart rate, wherein, in this case, the runtime difference to the electrical impulse in the heart which is resolved in the ECG measurement substantially depends on the blood pressure and, where applicable, on the condition of the blood vessels. If the condition of the blood vessels is known, further delays to the propagation time caused by this can thus be taken into account in the calculations by way of corresponding correction terms, such that it is additionally possible for the hearing system to measure blood pressure.

In this case, the hearing instrument of the hearing system preferably has an optical sensor for measuring the pulse of the wearer. Advantageously, the optical sensor contains at least one light source and one detection unit. An LED is preferably used as a light source, which is particularly preferably arranged in the hearing instrument in such a way that, when worn as intended, it is possible to emit a light into the skin of the ear canal. The light emitted from the LED is detected by the detection unit which is preferably formed as a photodiode. The signal which is detected by the optical sensor is preferably a voltage signal or current signal. Advantageously, a filter is connected downstream of the sensor, which filter serves to pre-filter a signal of the optical sensor for separating noise signals, interfering signals or background signals before the signal of the pulse measurement is delivered to the evaluation unit.

The hearing system preferably further contains a communications unit, wherein the evaluation unit is set up to output first warning information to the communications unit following a medically abnormal electrocardiogram, and wherein the communications unit is set up to transmit an emergency signal to medical personnel on the basis of the first warning information. In this case, the communications unit can be implemented, for example, by a mobile phone and a corresponding app for sending the emergency signal via 3/4/5G or the like, as well as, where applicable, for receiving the first warning information from an evaluation unit which is arranged in the hearing instrument. If the evaluation unit itself is also implemented by the mobile phone, a single app which provides all functions for the ECG and for transmitting the emergency signal as well as, where applicable, for the visualization of the ECG, is preferably recommended. Depending on how independent the wearer of the hearing instrument is, but also depending on an ascertained severity of the abnormality of the ECG, it is possible that the wearer is not or is no longer in a position to help themselves. In this case, transmitting the emergency signal, which, in addition to the medical data such as the ECG status, preferably also contains an optionally coded patient identification as well as GPS or similar localization data, can be life saving for the wearer.

A medically abnormal ECG is in particular intended to be understood here and hereinafter to mean an ECG which generally or also individually and specifically for the wearer of the hearing instrument has demonstratable, significant deviations which in particular represent a health threat or can result in a health risk. If a medically abnormal ECG of this type is registered by the evaluation unit, preferably based on a comparison with a number of reference ECGs which are saved in the evaluation unit, the evaluation unit outputs first warning information to the signal processing unit. In this case, if the evaluation unit is implemented in a remote control or a mobile phone, the warning information is wirelessly transmitted to the hearing aid and is correspondingly processed by a or the signal processing unit. This then adds the warning signal to the output signal which is the basis for the output sound which is delivered to the ear of the wearer, so that the wearer is informed about the abnormality of the ECG by way of the corresponding warning signal tone and can optionally consult a doctor or can take a medication to stabilize the heart activity.

Advantageously, the hearing instrument has at least one sound generator and one signal processing unit, wherein the evaluation unit is set up to output second warning information to the signal processing unit following a medically abnormal ECG, and wherein the signal processing unit is set up to output an output signal, which includes a warning signal, to the at least one sound generator on the basis of the second warning information. In this case, the at least one sound generator can be configured in particular as a speaker or as a bone conduction earphone. The output signal is converted into an output sound by the sound generator, which output sound is delivered to the ear of the wearer. In this case, the warning signal can be converted into a warning signal tone in the output sound, for example in the form of a distinctive beep or beep pattern. In this case, the second warning information can also be output to the signal processing unit irrespective of whether or not first warning information has been output to the or a communications unit of the hearing system.

The invention further refers to a hearing instrument with a first sensor electrode for generating a first sensor signal and a second sensor electrode for generating a second sensor signal, wherein an electrocardiogram of a wearer of the hearing instrument can be established using the first sensor signal and the second sensor signal if the hearing instrument is worn as intended by the wearer and the first sensor electrode and the second sensor electrode are each in contact with a body part of the wearer. The hearing instrument according to the invention shares the advantages of the hearing system according to the invention. In this case, the advantages of the developments of the hearing system can be transferred analogously to the hearing instrument. In this case, the hearing instrument is preferably configured as a hearing aid.

Other features which are considered as characteristic for the invention are set forth in the appended claims.

Although the invention is illustrated and described herein as embodied in a hearing system with a hearing instrument, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is an illustration of a hearing system set up for ECG measurement with a BTE hearing aid and a mobile phone according to the invention;

FIG. 2 is a diagrammatic, cross-sectional depiction of the hearing system set up for ECG measurement, which is implemented in an ITE hearing aid; and

FIG. 3 is a front view of a wearer of a hearing instrument, which is enabled by finger pressure on the housing for establishing an ECG.

### DETAILED DESCRIPTION OF THE INVENTION

Mutually corresponding parts and dimensions are each provided with the same reference numbers in all of the figures.

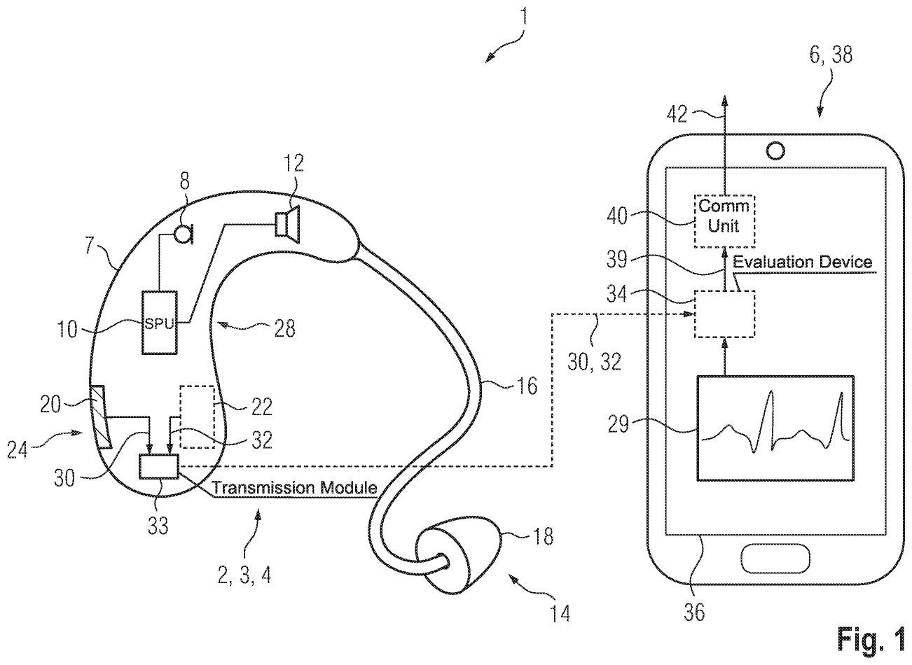

Referring now to the figures of the drawings in detail and first, particularly to FIG. 1 thereof, there is shown schematically a hearing system 1 with a hearing instrument 2 which is configured here as a hearing aid 3, specifically as a BTE aid 4, and a mobile phone 6, which are not reproduced to scale. The BTE aid 4 has a first input transducer 8 in a housing 7, which input transducer is configured as a microphone in the present case and is set up to generate an electrical input signal from an ambient sound. The input transducer 8 is connected to a signal processing unit 10 in the housing 7, which signal processing unit 10 processes the input signal generated by the input transducer 8 in a user-specific manner, i.e. in particular using the audiometric requirements of the wearer of the BTE aid 4, and amplifies it, in particular depending on the frequency band, in order to compensate for a hearing impairment of the wearer. In particular in the housing 7, the hearing aid 3 can optionally have even more input transducers which are not depicted in FIG. 1, the electrical input signals of which also being delivered to the signal processing unit 10 in order to be correspondingly processed.

The signal processing unit 10 is connected to a sound generator 12 which in the present case is configured as a speaker and converts a fully processed output signal of the signal processing unit 10 into an output sound. During operation of the hearing aid 3, the output sound is delivered to the ear of the wearer through an earpiece 14, which contains a sound tube 16 and an earmold 18, by the earmold 18 being inserted into the concha of the wearer in such a way that the outlet of the sound tube 16 leads directly to the entrance of their ear canal. For this purpose, the housing 7 of the BTE aid 4 is worn by the wearer behind their auricle during operation.

The BTE aid 4 has a first sensor electrode 20 and a second sensor electrode 22. In this case, the first sensor electrode 20 is arranged on the frame 24 of the housing 7, which frame faces away from the transition from the auricle to the head when the housing 7 is worn as intended behind the auricle, in such a manner that it is not in contact with the scalp, the auricle or any other body part of the wearer. The second sensor electrode 22 is incorporated into that lateral face 28 of the housing 7 which lies against the scalp of the wearer when worn. In FIG. 1, this lateral face 28 would be the lateral face of the housing 7 which faces away from the line of sight. The second sensor electrode 22 can advantageously also be applied to an inside curve on the underside of the housing 7. As a result, the second sensor electrode 22 can be pushed onto the skin when measuring for making better contact. One alternative configuration can also be realized in which the second sensor electrode is incorporated into the opposite lateral face of the housing 7 which lies against the auricle when the BTE aid 4 is worn (i.e. into the lateral face which faces the line of sight).

By means of the first sensor electrode 20 and the second sensor electrode 22, the wearer can use the hearing system 1 to establish an ECG 29 and to be able to have it displayed accordingly. For this purpose, if the wearer touches the point at which the first sensor electrode 20 is arranged in or on the housing 7 with a part of the hand, preferably with a fingertip or a knuckle or the like, a first sensor signal 30 is generated by the first sensor electrode 20. A second sensor signal 32 is correspondingly generated by the second sensor electrode 22.

In this case, the first sensor electrode 20 and the second sensor electrode 22 can be arranged on the outer surface of the housing 7 or can be incorporated into the material of the housing 7, preferably near the outer surface, in such a way that the corresponding electrical measurements which are necessary for the ECG 29 are possible. In an alternative which is not depicted, the second sensor electrode 22 can also be arranged on the earmold 18, wherein, in such a case, a sensor cable runs along the sound tube 16 in order to lead the second sensor signal to the housing 7.

In order to generate the first sensor signal 30 or the second sensor signal 32, a supply with an operating voltage, which is not depicted in greater detail, can be provided and set up for the first sensor electrode 20 or the second sensor electrode 22 in the BTE aid 4. In particular, the first sensor electrode 20 can in this case also be set up to register contact with a part of the hand of wearer, so that measuring the electrical activity on the heart of the wearer for establishing an ECG 29 is only initiated following contact of this type.

In order to generate the ECG 29, the first sensor signal 30 and the second sensor signal 32 are transmitted to the mobile phone 6 by way of a transmission module 33 in a suitable manner which is not described in greater detail, for example by means of Bluetooth or Near Field Communication or the like. In this case, the mobile phone 6 is set up for receiving the two sensor signals 30, 32 via communication protocol of this type. An evaluation unit 34 is implemented in the mobile phone 6 on the internal hardware by means of a corresponding app, which evaluation unit is set up to evaluate the first sensor signal 30 and the second sensor signal 32 and establish the ECG 29 therefrom.

The wearer can thus use the hearing system 1 formed from the hearing aid 3 and the mobile phone 6 to instantly obtain valuable medical information themselves in the form of the ECG 29, for which purpose they only need to initiate measuring and thus generating the sensor signals 30, 32 and has to touch the point of the first sensor electrode 20 on the housing 7 with a finger or the like in order to achieve an advantageous electrical circuit on the body. The ECG 29 can also be initiated by a corresponding command in the app.

Furthermore, the mobile phone 6 forms a visualization means 38 for the ECG 29. For this purpose, the app is configured to graphically display the ECG 29 established and provided by the evaluation unit 34 via a screen 36 of the mobile phone 6, so that the wearer can recognize abnormal values instantly. The evaluation unit 34 implemented by the app can independently recognize particularly abnormal ECG 29 values or ECG values that are even threatening to the wearer's health. In this case, the evaluation unit 34 outputs first warning information 39 to a communications unit 40 of the mobile phone 6, such that the communications unit 40 emits an emergency signal 42 which can be received by a doctor, a hospital, an emergency medical service or the like, in order to be able to provide the wearer with medical assistance immediately.

In this case, the communications unit 40 is implemented by means of the native components which are provided in the mobile phone 6 for transmitting signals to a mobile network or a mobile data network (e.g. antennas for 3/4/5G communication and the like), wherein, for example, the first warning information 39 may be sent as the emergency signal 42 from the components, in other words the app generates the first warning information 39 already in a data format and with all the information which the receiver of the emergency signal 42 requires in order to treat the wearer 42 as quickly as possible, and the communications unit 40 transmits this information as the emergency signal 42. Other implementations are conceivable. In this case, the emergency signal 42 preferably also contains GPS data and/or similar information for localization of the wearer.

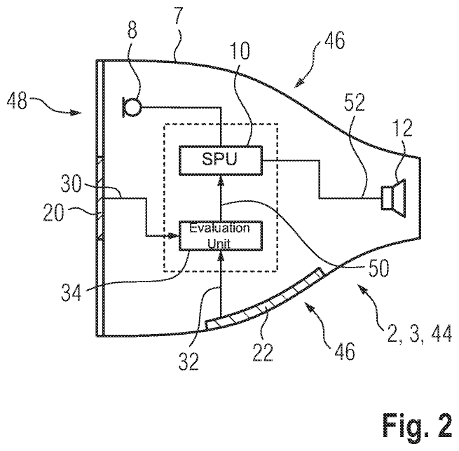

FIG. 2 shows a cross-sectional depiction of a hearing system 1 which is fully implemented in a hearing aid 3 which is configured as an ITE aid 44. In order to wear the ITE aid 44 as intended, the aid is partially inserted into the ear canal of the wearer. For this purpose, the housing 7 has a molding 46 which is modeled on the outer ear canal and is preferably individually adjusted to the anatomy of the wearer. The housing of the ITE aid 44 contains a cover plate 48 (so called “faceplate”; perpendicular to the line of sight) into which the first sensor electrode 20 is incorporated in such a way that it faces the free space next to the ear when the ITE aid 44 is worn. The second sensor electrode 22 is incorporated into the molding 46 on the housing. In this case, the evaluation unit 34 is arranged in the housing 7 and can in particular be implemented on the same system on a chip 49 on which the signal processing unit 10 is also implemented.

For an ECG, the wearer can now touch, with a finger or the like, the cover plate 48 at the point over which the first sensor electrode 20 extends. From the moment that the ITE aid 44 is worn, the second electrode 22 is in constant contact with the skin of the wearer. The first sensor signal 30 and the second sensor signal 32 are evaluated in the evaluation unit 34 in the same manner as in the exemplary embodiment described using FIG. 1, with the difference being that in the present case the evaluation unit is arranged in the housing 7 and thus the sensor signals 30, 32 do not have to be transmitted to an external unit, such as the mobile phone 6. Similarly to FIG. 1, the visualization of the generated ECG can take place in an external visualization means (not depicted) which can be implemented in a mobile phone or an individual remote control of the ITE aid 44.

In the event of a medically abnormal ECG, the evaluation unit 34 can output second warning information 50 to the signal processing unit 10—e.g. via a corresponding interface—so that a warning signal is fed into the output signal 52 which is output to the sound generator, which warning signal is also output by the sound generator 12. The wearer can then hear this warning signal and is thus informed about the abnormality so that they can consult a doctor, a hospital, an emergency medical service or the like.

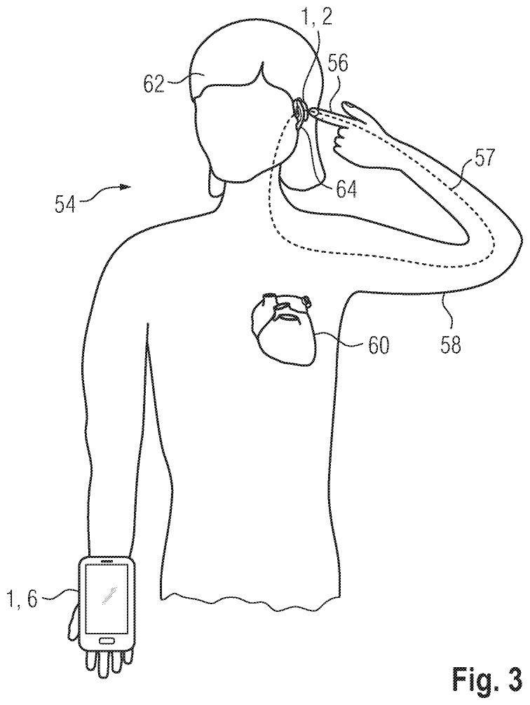

FIG. 3 schematically depicts a wearer 54 of a hearing instrument 2. In this case, the hearing instrument 2 can be configured as a hearing aid, for example as the BTE aid 4 according to FIG. 1 or as the ITE aid 44 according to FIG. 2, or it can be represented by an earphone. In a manner which is not depicted in greater detail, the hearing instrument 2 has a first sensor electrode and a second sensor electrode for establishing an ECG, and it is part of the hearing system 1 which moreover contains the mobile phone 6. In order to establish an ECG with the hearing system 1 as described above, the wearer 54 touches the housing of the hearing instrument 2 with a finger 56 at the point at which the first sensor electrode is arranged. As a result, an electrical circuit 57 is closed on the body of the wearer 54, which circuit leads to the head 62 of the wearer via the arm 58, past the heart 60 and thus to the ear 64 where the second sensor electrode touches the skin of the wearer. This makes it possible to measure the electrical activity of the heart 60 using the sensor signals of the two sensor electrodes, without too great an influence from other electrical interference signals, and to establish an ECG therefrom.

Although the invention has been illustrated and described in greater detail by way of the preferred exemplary embodiment, the invention is not limited by the disclosed examples and other variations can be derived therefrom by the person skilled in the art without departing from the scope of protection of the invention.

### LIST OF REFERENCE NUMBERS

1 hearing system
2 hearing instrument
3 hearing aid
4 BTE aid
6 mobile phone
7 housing
8 input transducer
10 signal processing unit
12 sound generator
14 earpiece
16 sound tube
18 earmold
20 first sensor electrode
22 second sensor electrode
24 frame
28 lateral face
29 ECG/electrocardiogram
30 first sensor signal
32 second sensor signal
33 transmission module
34 evaluation unit
36 screen
38 visualization means
39 first warning information
40 communications unit
42 emergency signal
44 ITE aid
46 molding
48 cover plate
49 system on a chip
50 second warning information
52 output signal
54 wearer
56 finger
57 electrical circuit
58 arm
60 heart
62 head
64 ear

1. A hearing system, comprising:
a hearing instrument having a first sensor electrode, a second sensor electrode and an evaluation unit, wherein said evaluation unit is set up to establish an electrocardiogram (ECG) of a wearer of said hearing instrument using a first sensor signal, which is output by said first sensor electrode, and a second sensor signal, which is output by said second sensor electrode, if said hearing instrument is worn as intended by the wearer and said first sensor electrode and said second sensor electrode are each in contact with a body part of the wearer.

2. The hearing system according to claim 1, wherein said first sensor electrode is disposed in said hearing instrument in such a way that it is readily accessible when worn as intended.

3. The hearing system according to claim 2, wherein said evaluation unit is set up to establish the electrocardiogram of the wearer if the wearer touches with a part of a hand a point on said hearing instrument at which said first sensor electrode is disposed.

4. The hearing system according to claim 1, further comprising a visualization means, wherein said evaluation unit is set up to transmit an established electrocardiogram to said visualization means, and wherein said visualization means is set up to graphically display a received electrocardiogram.

5. The hearing system according to claim 4, wherein said evaluation unit is incorporated into said visualization means.

6. The hearing system according to claim 1, wherein said evaluation unit is incorporated into said hearing instrument.

7. The hearing system according to claim 1, wherein said hearing instrument is configured as an earphone.

8. The hearing system according to claim 1, wherein said hearing instrument is configured as a hearing aid.

9. The hearing system according to claim 1, wherein said hearing instrument further comprises an earpiece, and wherein said second sensor electrode is arranged in said earpiece.

10. The hearing system according to claim 8, wherein said hearing instrument further has a housing which is set up to be worn behind an auricle of the wearer, and wherein said second sensor electrode is disposed in said housing.

11. The hearing system according to claim 1, wherein;
said hearing instrument is further set up to measure a pulse of the wearer; and
said evaluation unit is set up to determine a blood pressure using a measured pulse and the ECG.

12. The hearing system according to claim 11, wherein said hearing instrument has an optical sensor for measuring the pulse of the wearer.

13. The hearing system according to claim 1, further comprising a communications unit, wherein said evaluation unit is set up to output first warning information to said communications unit following a medically abnormal electrocardiogram, and wherein said communications unit is set up to transmit an emergency signal to medical personnel on basis of the first warning information.

14. The hearing system according to claim 1, wherein:
said hearing instrument has at least one sound generator and one signal processing unit;
said evaluation unit is set up to output second warning information to said signal processing unit following a medically abnormal electrocardiogram; and
said signal processing unit is set up to output an output signal, which includes a warning signal, to said at least one sound generator on a basis of the second warning information.

15. A hearing instrument, comprising:
a first sensor electrode for generating a first sensor signal; and
a second sensor electrode for generating a second sensor signal, wherein an electrocardiogram of a wearer of the hearing instrument can be established using a first sensor signal and a second sensor signal if the hearing instrument is worn as intended by the wearer and said first sensor electrode and said second sensor electrode are each in contact with a body part of the wearer.
